(19) United States
(12) Patent Application Publication
(10) Pub. No.: US 2015/0328284 A1
(43) Pub. Date: Nov. 19, 2015

(54) COMPOSITIONS AND METHODS COMPRISING TOLL LIKE RECEPTOR (TLR) STIMULATING AGENTS FOR PROPHYLAXIS AND THERAPY FOR DAMAGE TO DERMAL EPITHELIUM

(71) Applicant: Cleveland BioLabs, Inc, Buffalo, NY (US)

(72) Inventors: Andrei GUDKOV, East Aurora, NY (US); Lyudmila BURDELYA, Lancaster, NY (US)

(21) Appl. No.: 14/389,862

(22) PCT Filed: Apr. 2, 2013
(86) PCT No.: PCT/US2013/034942

§ 371 Date: Oct. 1, 2014

Related U.S. Application Data
(60) Provisional application No. 61/619,362, filed on Apr. 2, 2012.

(51) Int. Cl. A61K 38/16; A61K 9/70

(52) U.S. Cl. CPC A61K 38/164; A61K 9/70

(57) ABSTRACT

Provided are compositions and methods for prophylaxis and/or therapy of dermal damage. The compositions and methods involve Toll like receptor (TLR) agonists that can inhibit the formation or accelerate healing of dermal damage, and/or inhibit inflammation at near the location of the dermal damage. The TLR agonists can be TLR2 and/or TLR5 agonists. The compositions and methods are suitable for prophylaxis and/or therapy of dermal damage caused by radiation, or non-radiation thermal injury such as burns caused by contact with heated surfaces or heated objects, light, and for disruptions in integument resulting in cuts, such as incisions, or punctures. The compositions can be used topically, subcutaneously or systemically. Articles of manufacture coated or impregnated with TLR agonist(s) are also provided.

### CROSS REFERENCE TO RELATED APPLICATIONS

This application claims priority to U.S. Provisional application No. 61/619,362, filed on Apr. 2, 2012, the disclosure of which is incorporated herein by reference.

### FIELD

The present disclosure relates generally to dermal injury and more particularly to compositions and methods for prophylaxis and therapy of dermal injuries.

### BACKGROUND

Cutaneous radiation damage occurs frequently in cancer patients during local radiotherapy which curtails its application and thereby limits its effectiveness. Tissue stem cells are the most critical component in the protection of radiosensitive tissues as these cells determine the ability of an injured organ to regenerate. There is an ongoing need to provide compositions and methods to inhibit and cutaneous injures resulting from radiation therapy, as well as other thermal injuries and wounds such as those resulting from surgical procedures and via contact with heated objects or surfaces. The present disclosure addresses these needs.

### SUMMARY OF THE DISCLOSURE

The present disclosure describes compositions and methods for prophylaxis and/or therapy of dermal damage. The compositions and methods involve Toll like receptor (TLR) agonists that can inhibit the formation or accelerate healing of dermal damage, and/or inhibit inflammation at near the location of the dermal damage. The TLR agonists include TLR2 and/or TLR5 agonists.

The compositions and methods are suitable for prophylaxis and/or therapy of dermal damage caused by any of radiation, or non-radiation thermal injury, including but not necessarily limited to burns caused by contact with heated surfaces or objects, or light, and for disruptions in the integument of an individual, such as cuts, medical incisions and punctures. The compositions can be used topically, subcutaneously or systemically. Articles of manufacture coated or impregnated with TLR agonist(s) are also provided.

In one approach, the present disclosure provides a method for prophylaxis and/or therapy of dermal damage comprising administering to an individual a composition comprising a TLR agonist, wherein the administration is performed prior to, concurrent with or subsequent to the dermal damage. In embodiments, the method of administration is selected from topical, subcutaneous, or systemic administration. The compositions and methods can include a TLR agonist elected from a TLR2 agonist, a TLR5 agonist, and a combination thereof. In particular embodiments, the TLR5 agonist is the known compound, CBLB502, and the TLR2 agonist is the known compounds CBLB612 or CBLB613. In embodiments, the dermal damage addressed by the present disclosure is a result of radiation, or non-radiation thermal damage, or a cut, medical incision or puncture.

The disclosure also includes pharmaceutical, cosmetic and nutraceutical formulations that are suitable for topical or subcutaneous administration, and which comprise one or a combination of TLR agonists.

In embodiments, the disclosure also provides articles of manufacture comprising a dermal damage healing aid, wherein the dermal damage healing aid is coated and/or impregnated with a TLR agonist. In embodiments, dermal damage healing aid is coated with a TLR2 agonist, a TLR5 agonist, or a combination thereof. In embodiments, the dermal damage healing aid is selected from a medical dressing, a suture, a bandage, and a fabric mesh.

### DESCRIPTION OF THE FIGURES

FIG. 1A provides a photographic representation of results of mice and skin sections from neck area of mice irradiated 3×10 Gy without or with CBLB502 which were taken 14 days after first irradiation (IR) for morphological evaluation of tissue damage using hematoxylin/eosin staining (H&E). FIG. 1B provides photographic representations of samples of dorsal and ventral oral epithelium obtained from mice 11 days after fractioned 30 Gy IR without or with CBLB502 (H&E). Samples obtained from an intact mouse was used as a control (u/t).

FIG. 2 provides photographic representations of representative burns from CBLB502 and CBLB613 treated mice and untreated control.

FIG. 3 provides photographic representations of wounds of CBLB502 treated mice and untreated mice taken days 8 after thermal wounding (6 mice/group).

FIG. 4. Graphical summaries of neutrophil and macrophage activity in skin after 50 Gy of X-ray IR estimated by the intensity of luminol and lucigenin in CBLB502 (n=6) and PBS (n=4) injected mice using IVIS Imaging System and Living Image software (Caliper LifeSciences).

FIG. 5. Photographic and graphical representations of data pertaining to the damaging effect of 50 Gy in skin 52 days after IR. Representative images and quantitation of bioluminescence intensity obtained using lucigenin in CBLB502 and PBS injected mice (n=6).

FIG. 6. Graphical representation of results obtained from FACS analysis of total leukocytes (CD45+), neutrophils (CD45+CD11b+GR-1hi) and macrophages (CD45+CD11b+F4/80+) in sponges inserted into incisions of mice treated with PBS or CBLB502 (1 μg, s.c.) for the indicated amounts of time (hs). The absolute number of cells per sponge is indicated, mean±SEM, 4 mice/group.

### DETAILED DESCRIPTION

The present disclosure relates generally to prophylaxis and therapy for dermal damage. “Dermal damage” as used herein means damage to any one or any combination of the dermis, epidermis, basement membrane and hypodermis, and thus can include any disruptions of mammalian integument. Dermal damage can also include damage to mucous membranes, the tongue and lips, and any other dermal epithelium. Dermal damage that is a subject of this disclosure may be or may not be accompanied by damage to other body tissue, including but not limited to muscle, vasculature, tendons, ligaments, bones and organs. In non-limiting embodiments compositions and methods provided in this disclosure promote and/or enhance regeneration of damaged skin and/or wound healing and/or inhibit inflammation at or near the location of the dermal damage.

In general, embodiments comprise contacting the dermis and/or otherwise introducing into an individual a composition comprising Toll-like receptor (TLR) agonist in a formulation effective to inhibit the formation of or treat dermal damage.

In embodiments, the TLR stimulating agent is a TLR2 or TLR5 agonist. Combinations of such agonists can also be used. In various embodiments, a TLR5 agonist is used.

Dermal damage addressed by the present disclosure includes but is not necessarily limited to damage caused by radiation, such as radiation used during cancer therapy or other medical interventions, or other thermal injury such as burns, including but not necessarily limited to burns induced by contact with heated objects and/or surfaces, or light, including but not-limited to sunlight, or light used in medical procedures such as photodynamic therapy, dermal damage caused by chemicals, and dermal damage caused by medical techniques such as surgical interventions wherein the skin is cut or pierced or avulsed, or other non-medical wounds which cause dermal trauma by any means. In embodiments, the disclosure pertains to a circumstance where the skin of an individual is cut, wherein the cut can include but is not necessarily limited to an incision made with a sharp object, such as a scalpel or other cutting implement. In embodiments, the dermal damage which is inhibited and/or treated using compositions and methods of this disclosure is created intentionally or is incidental to a medical procedure. In other embodiments the dermal damage is not created by or during a medical procedure, but results from another source of dermal trauma, such as accidental contact with a heated surface or chemical, or accidental exposure to radiation, or an accidental or an intentional non-medical circumstance whereby the dermis is cut or punctured or avulsed, including but not necessarily limited to dermal trauma resulting from participation in sporting events, industrial accidents, household accidents, and acts of violence.

In embodiments, the present disclosure provides cosmetic or pharmaceutical or nutraceutical compositions comprising a cosmetically or pharmaceutically or nutraceutically effective amount of any one or any combination of the TLR agonists described herein. The compositions can be produced in any solid, liquid or semisolid formulation and can include acceptable carriers with formulation ingredients such as salts, carriers, buffering agents, emulsifiers, diluents, excipients, chelating agents, fillers, drying agents, antioxidants, preservatives, binding agents, bulking agents, silicas, solubilizers, or stabilizers, or any combination thereof. Examples of acceptable carriers can be found in: Remington: The Science and Practice of Pharmacy (2005) 21st Edition, Philadelphia, Pa. Lippincott Williams & Wilkins. The preparations can formulated to be administered using any acceptable route.

In embodiments, the compositions and methods of the disclosure are suitable for prophylaxis and/or therapy of any human or non-human animal, such as any mammal, including human and non-human mammals, and is thus considered in embodiments to be pertinent for use in human and veterinary medicine.

Compositions provided by this disclosure include but are not necessarily limited to creams, aqueous solutions, suspensions or dispersions, oils, balms, foams, lotions, gels, cream gels, hydrogels, liniments, serums, films, ointments, sprays or aerosols, or any multiple emulsions, slurries or tinctures. The compositions can also include liposomes, microsomes, nanoparticles, and any other suitable vehicle for delivering a TLR agonist to or through some or all the layers of the skin of an individual. The compositions can be used for prophylaxis and/or therapy of dermal damage that is localized or is more widespread over the skin of an individual.

The compositions can comprise any additional agents intended to directly or incidentally assist with inhibition and/or treatment of dermal damage, such as antibiotics, growth factors, collagen, vitamins, plant extracts, sunscreens, lipids, fatty acids, transdermal penetration enhancers, or any other agent intended to enhance delivery and/or stability and/or efficacy of the composition. The compositions can include or be administered with a topical anaesthetic agent.

Pharmaceutical or cosmetic formulations of the present disclosure can be made using any suitable techniques and can be provided in containers or delivery devices, including but not limited to vials, syringes, wipes, adhesive patches, non-adhesive patches, bandages, wound dressings, compresses, fabric meshes, sutures, or any other product or device intended to treat, close, protect or otherwise inhibit the formation of or promote healing of dermal damage.

In embodiments, the present disclosure comprises articles of manufacture that comprise one or more of the TLR agonists described herein. In non-limiting embodiments, the articles of manufacture include but are not limited to adhesive patches, non-adhesive patches, bandages, medical dressings (i.e., wound dressings and/or surgical dressings), compresses, fabric meshes, sutures, or any other product or device that is used to treat, close, protect or otherwise inhibit the formation of or promote healing of dermal damage (collectively “dermal damage healing aids”) can be coated with a composition comprising a TLR agonist described herein, and/or can have the TLR agonist integrated into some or all of the material of which the dermal damage healing aid is made. In embodiments, a medical dressing is a component of a bandage. In embodiments, the present disclosure includes a TLR-modified dermal healing aid that comprises a first material, wherein the first material comprises cotton, silk, paper, a polymer, or a combination thereof, and a second material, wherein the second material comprises a TLR agonist, wherein the TLR agonist is coated onto the first material and/or is impregnated into the first material. In embodiments, the TLR containing composition can be coated onto a surface of the dermal damage healing aid that is intended to be in contact with a wound and/or surgical incision. Such surface(s) of the dermal damage healing aid in certain embodiments may be an absorbent material.

Coating and/or impregnating the dermal damage healing aids can be performed using techniques well known to those skilled in the art, including but not necessarily limited to spraying, dipping, soaking, powdering, drying, baking and dry-coating, and combinations thereof.

For non-topical administration, any device and/or technique for administration can be used. In embodiments, the composition comprising a TLR agonist is administered subcutaneously (s.c.), intravenously (i.v.), intramuscularly (i.m.), intraperitonealy (i.p.), or orally. In embodiments, the composition comprising a TLR agonist is administered s.c. prior to or subsequent to dermal damage.

Dosages of the TLR agonist can be determined, given the benefit of the present disclosure, by those skilled in the art and will depend on well know factors such as the type and degree of dermal damage, and the route of administration. In embodiments, from 0.1 to 1.0 mg/kg body weight, inclusive, and including all integers and ranges between 0.1 to 1.0 to the first decimal point, can be used. In one embodiment, from 0.2-0.5 mg/kg can be used. In one embodiment, 0.2 mg/kg body weight can be used. In embodiments, formulation for s.c. administration is provided at 1 μg/0.1 ml.

The agents can be administered prior to, concurrently or subsequent to dermal damage. The administration can be a single administration, or can comprise a series of administrations. The administrations can be one or more times per day, including up to eight times per day, such as at least four time per day, and continued for a period of days, and can be administered in any intervals, such as once every two, three, four, five or six days, or once a week. A series of administrations can be performed for at least two days, and over a period of weeks, months or years. In embodiments, the method results in any one or any combination of i) increased rate and/or degree of wound healing/skin regeneration; ii) decreased dermal damage; iii) reduced inflammation in conjunction with dermal damage, which can be evidenced by a reduction in any well-known marker(s) of inflammation, including but not necessarily limited to a reduction in number and/or activity of neutrophils and/or macrophages at or near the site of the damage. In embodiments, use of the compositions and methods of this disclosure can result in inhibition of scar tissue formation. Use of the compositions and methods of the invention can be compared to any suitable reference to ascertain the degree of prophylaxis and/or therapeutic effect in connection with dermal damage.

In embodiments, the present disclosure comprises articles of manufacture comprising the TLR agonists. The article of manufacture can comprise packaging, which may comprise printed information. The printed information can be provided on a label, or on a paper insert, or printed on the packaging material itself. The printed information can include information that identifies the TLR agonists in the package, an indication of what condition the agonists are intended to treat, and instructions for using the compositions, such as the amount of the composition to use, the route of administration, and the number of doses to take over a given period of time. In other embodiments, the article of manufacture comprises a dermal damage healing aid that is coated and/or impregnated with a TLR agonists.

In one embodiment, the article of manufacture includes a TLR agonist provided in sealed container, along with packaging and printed information that describes using the composition comprising the TLR agonist for use in prophylaxis and/or therapy of dermal damage. In one embodiment, the printed material describes using the composition for prophylaxis and/or therapy of dermal damage caused by radiation. In another embodiment, the printed material describes using the composition for the prophylaxis and/or therapy of dermal damage for non-radiation induced thermal injury, such as by contact with a heated object and/or surface. In another embodiment, the printed material describes using the composition for prophylaxis and/or therapy of dermal damage from a surgical intervention, or dermal damage that involves disruption of the integrity of skin from a non-surgical intervention, and/or for inhibiting inflammation associated with creation and/or treatment of an open wound. The printed material may describe combinations of such uses, or only one of such uses. In embodiments, the printed material may identify the article of manufacture as a dermal damage healing aid, and may provide instructions on using the dermal damage healing aid for use in prophylaxis and/or therapy of dermal damage, such as for protecting and/or wound healing, and may identify TLR agonist(s) with which the dermal damage healing aid may be coated and/or impregnated.

In embodiments, the TLR agonist included in a composition for use in the methods disclosed herein comprises a TLR5 agonist, a TLR2 agonist, or a combination thereof. In connection with this, we have demonstrated that the TLR5 agonist known in the art as CBLB502, which is derived from bacterial flagellin, protects rodents and primates from doses of radiation that induce lethal acute radiation syndrome while, at the same time, offers no protection to tumor cells (Burdelya et al., Science, 2008). The structure of CBLB502 is known and is described in the art, such as in U.S. Pat. No. 8,287,882, from which the description of CBLB502 is incorporated herein by reference. We have also demonstrated radioprotective and radiomitigating activities for the TLR2 agonists CBLB612 and CBLB613, which are synthetic mimics of lipopeptides of mycoplasma, the structures of which are known in the art (see, for example, U.S. Pat. No. 8,008,260, from which the description of CBLB612 and CBLB613 is incorporated herein by reference; and Singh et al., Radiat Res May; 2012; Epub 2011 Dec. 16; 177(5):628-42).

Without intending to be constrained by theory, it is believed that the TLR agonists act by mobilizing a combination of natural tissue protection mechanisms largely mediated by induction of the pro-survival NF-kB pathway which results in up-regulation of endogenous antioxidants and anti-apoptotic and regeneration-inducing factors as well as activation of the innate and adaptive immune response.

In the present disclosure, we tested the effect of TLR5 agonists in the context of various types of skin injury. Our data demonstrate that compositions comprising CBLB502 significantly reduces the severity of radiation induced dermatitis and mucositis in mice caused by localized single or fractional radiation treatment and accelerates the recovery of irradiated animals. In other embodiments, we demonstrate that CBLB502 and CBLB612 treatment accelerated regeneration of damaged skin and wound healing in mice with local burns, and a composition comprising BLB502 can be used to inhibit inflammation during the treatment of open wounds.

The following Examples are intended to illustrate specific embodiments but are not intended to limit the present disclosure.

### Example 1

The following materials and methods were used to produce the results presented in the Examples and figures.

CBLB502 Injection and Irradiation.

NIH-Swiss mice were injected with PBS or CBLB502, s.c. 1 μg/mouse for fractioned and 2.5 μg/mouse for single radiation treatment 30 min prior to irradiation. For head and neck model, head and neck areas were irradiated with 10, 15, 20 or 25 Gy X-ray IR under isofluorane inhalation anesthesia. Mouse body weight and survival were recorded daily or every second day. For IHC analysis, mice were sacrificed 11 or 14 days after IR. For a single leg irradiation model, mouse hind limbs were exposed to 50 Gy X-ray IR applied in two daily 25 Gy doses (as described in Kumar et al, 2008, Radiat Oncol 3:40). Mice were injected s.c. with either CBLB502 (0.2 mg/kg mouse body weight) or PBS vehicle 1 h after each IR followed by 3 daily injections (5 total injections).

Assessment of Inflammation.

The degree of inflammation and its resolution was assessed using non-invasive imaging of live mice which provides quantitative measurements of luminol bioluminescence generated by myeloperoxidase activity of neutrophils and lucigenin bioluminescence induced by NADPH oxidase activity of macrophages (as described in Tseng and Kung, 2012, Chemistry & Biology, 19:1199-1209).

Burn Wounding and Treatment.

The dorsal region of female BALB/c mice (NCI) was shaved and depilated at least 48 hours before thermal wounding. Mice were anesthetized by i.p. injection of ketamine:xylazine (100 mg/kg:10 mg/kg). A 1.0-cm diameter brass rod (104 g with a 33 g Teflon extension) was heated in a 100° C. circulating water bath for 2 minutes and then placed in contact with the posterior dorsum of each mouse for 20 seconds. The rod was held in place by a custom-made positioner with a constant force of 1.34 Newtons. Immediately following wounding, mice were rehydrated with s.c. saline. Buprenorphine was given (s.c., 0.05 mg/kg) for post-wound analgesia. One hour after wounding, mice were injected with CBLB502 or CBLB612 (s.c. 1 μg/0.1 ml/mouse) followed by 4 additional daily injections. Endpoints are wound size as a function of time after wounding (using digital planimetry), escar adhesion and histological assessment.

Incisional Wounding (Cuts) and Treatment.

The dorsal region of female NIH Swiss mice (NCI) was shaved at least 48 hours before incisional wounding. Mice were anesthetized using isofluorane inhalation anesthesia. A cut about 1.0-cm long was made using sterile scalpel. Sterile PVA research sponges were inserted and wounds were closed using wound clips Immediately (<10 min) after the wounding procedure, mice were injected with CBLB502 (s.c. 1 μg/0.1 ml/mouse) or PBS as a vehicle control. Mice were sacrificed and sponges removed 24 h, 72 h and 120 h post treatment, 4 mice per each treatment and time-point. Cells attracted to the sponges were isolated by PBS was and analyzed by FACS for cell specific marker to determine total leukocyte (CD45⁺), neutrophil (CD45⁺CD11b⁺GR-1^{hi}) and macrophage (CD45⁺CD11b⁺F4/80⁺) cell number/sponge.

Histology. H&E staining of sections of tongues, lips and skin samples from the front neck area of irradiated and non-irradiated control mice was performed and analyzed ‘blindly’ at 20× magnification.

### Example 2

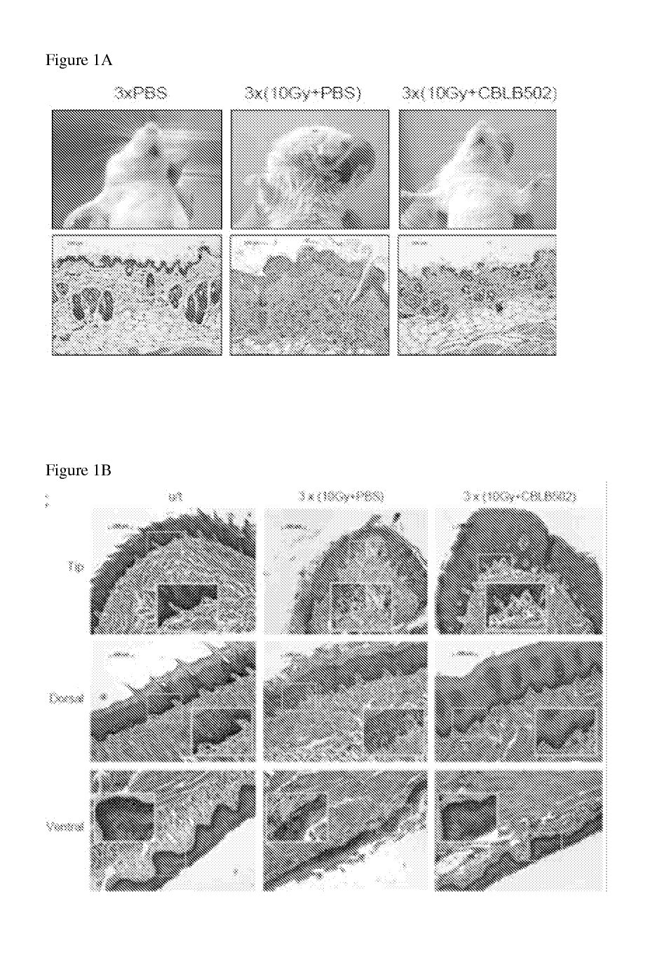

This Example demonstrates radioprotective effect of CBLB502 on skin and tongue epithelium during radiation treatment of head and neck area. Representative results are depicted generally in FIGS. 1A and 1B. FIG. 1A provides a photographic representation of results of mice and skin sections from neck area of mice irradiated 3×10 Gy without or with CBLB502 which were taken 14 days after first irradiation (IR) for morphological evaluation of tissue damage using hematoxylin/eosin staining (H&E). FIG. 1B provides photographic representations of samples of dorsal and ventral oral epithelium obtained from mice 11 days after fractioned 30 Gy IR without or with CBLB502 (H&E). Samples obtained from an intact mouse was used as a control (u/t).

As can be seen from FIGS. 1A and 1B, samples of skin from the irradiated neck area and dorsal and ventral tongue epithelium without prior treatment with CBLB502 displayed severe damage to the dermal and mucosal epithelium (atrophy of the hair follicles, hemorrhage, erosion, ulceration, hyperplasia, dysplasia and infiltration of the inflammatory cells). By contrast, those mice treated with CBLB502 exhibited minor or no tissue damage.

### Example 3

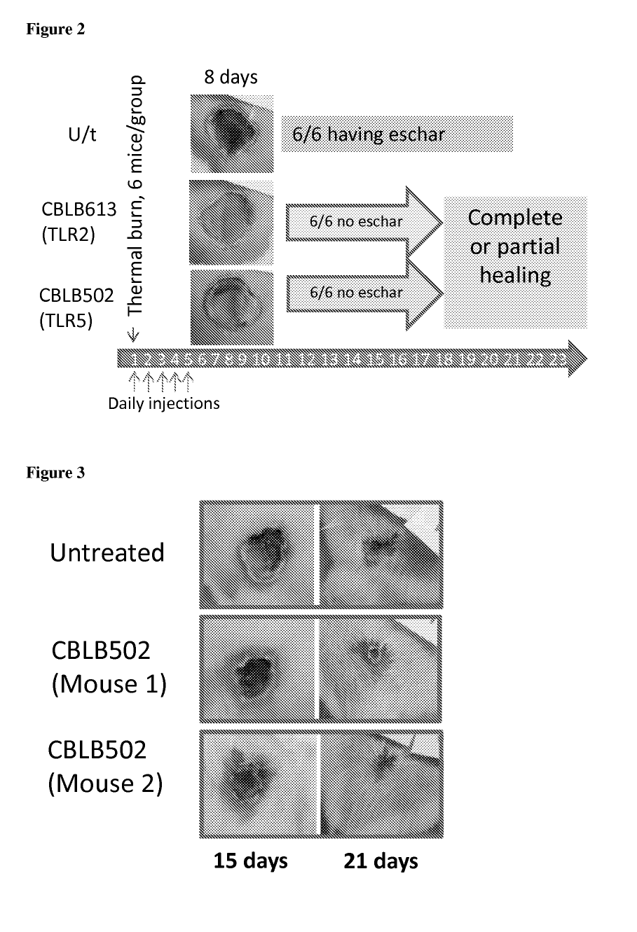

This Example demonstrates acceleration of burn wound healing by TLR5 (CBLB502) and TLR2 (CBLB613). The results of the TLR agonist therapy comprise faster scab removal and skin regeneration in the treated mice in comparison with untreated control mice (6 mice/group-treatment). In particular, FIG. 2 provides photographic representations of representative burns from CBLB502 and CBLB613 treated mice and untreated control. Shedding eschars (scabs) from burned areas is indicative of faster regeneration of skin after thermal wounding. A significant difference in the healing of the burn injuries was observed on day 11 when all eschars were loose or shed off in all CBLB502 and CBLB613 treated mice, whereas they remained in the untreated control mice. By day 18, the CBLB502 and CBLB613 treated mice exhibited partial or complete healing with significantly smaller wound areas with more regenerated tissues surrounding the wounds than untreated control mice.

### Example 4

This Example summarizes representative dynamics of skin burn wound healing in CBLB502 treated mice. To obtain the results presented in FIG. 3, we analyzed the progress of wound healing at different time points during a three week period after treatment application. FIG. 3 provides images of representative wounds of CBLB502 treated mice and untreated mice taken days 8 after thermal wounding (6 mice/group). By day 8, eschars (scabs) formed on the burned areas of the CBLB502 treated mice were loose and partially shed off (4/6 mice) in contrast to the eschars still firmly attached to the burns of all untreated control mice. By day 15, all eschars were shed off from the burns of the CBLB502 (6/6) treated mice; those of the control group remained in place. By day 21, the CBLB502 treated mice exhibited significantly smaller wound areas with more regenerated tissues surrounding the wounds than untreated control mice.

### Example 5

This Example demonstrates radioprotective and mitigating effects of CBLB502 on skin epithelium applied after dermal damaging radiation exposure. Mouse hind limbs were exposed to 50 Gy X-ray IR applied in two daily 25 Gy doses (as described in Kumar et al, 2008, Radiat Oncol 3:40). Mice were injected s.c. with either CBLB502 (0.2 mg/kg mouse body weight) or PBS vehicle 1 h after each IR followed by 3 daily injections (5 total injections).

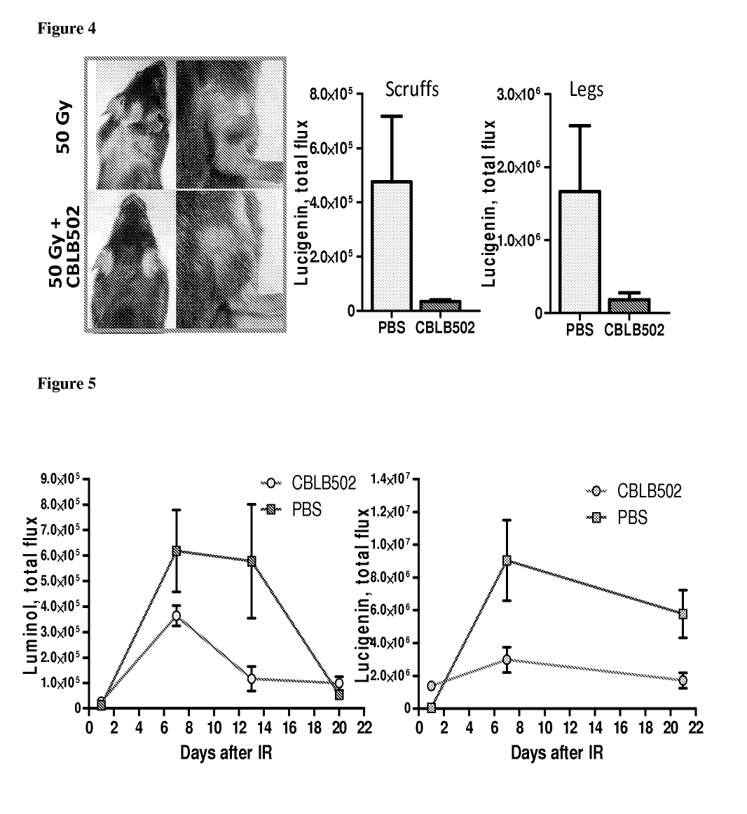

As summarized by the graphs presented in FIG. 4, development of an inflammatory response in the irradiated skin was first observed ˜7 days after IR as redness followed by increasing inflammation, ulceration and moist desquamation. Non-invasive imaging of live mice using luminol and lucigenin bioluminescence detecting activated neutrophil and macrophage accumulation revealed significantly less neutrophil and macrophage activity (indicative of less inflammation) during the first 1-3 weeks after IR in the skin of irradiated mice treated with CBLB502 as compared to those treated with PBS.

### Example 6

This Example demonstrates inhibition and mitigation late (delayed) radiation damage to skin. Mouse hind limbs were exposed to 50 Gy X-ray IR applied in two daily 25 Gy doses. Mice were injected s.c. with either CBLB502 (0.2 mg/kg mouse body weight) or PBS vehicle 1 h after each IR followed by 3 daily injections (5 total injections). Mice were observed up to 2 months post-IR. As summarized in FIG. 5, delayed radiation damage was observed starting approximately 30-40 days after IR with severe local irradiated ulceration and distant skin damage developed on the scruffs of all PBS-treated mice. In contrast, there was significantly less damage in the irradiated skin and no or minimal damage in distant areas found in CBLB502-treated mice.

### Example 7

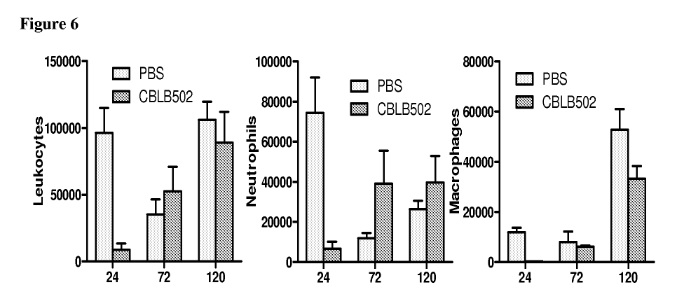

This Example demonstrates anti-inflammatory activity of CBLB502 in an incisional skin wounding model (open cuts). Surgical incisional wounds (about 1 cm length) were made in the backs of mice. Sponges were inserted into incisional wounds (as described in Schaffe et al, 2007, J Surg Res 138:100-105). We performed FACS analysis of immune cells accumulated in the sponges at 24, 72 and 120 hours after the wounding. As can be seen from the results depicted in FIG. 6, CBL502 significantly suppressed leukocyte accumulation in skin after wounding in the incisional cut model. FACS analysis of immune cell content in sponges inserted into incisional wounds showed that PBS-treated mice developed classic neutrophil influx during the first hours after wounding, with subsequent accumulation of macrophages. In comparison, CBL502-treated mice displayed significant reduction of total leukocytes, granulocytes and macrophages mobilized to the wound area (sponges). These data demonstrate that CBLB502 inhibits and suppresses inflammation in wounded skin, such as open cuts. These data also indicate that the mechanism of CBLB502 radioprotective/mitigating activity through reduction of sterile inflammation developed in the irradiated areas, which inhibits damage caused by reactive oxygen species and other factors produced by activated leukocytes in response to irradiation.

While the invention has been described through specific embodiments, routine modifications will be apparent to those skilled in the art and such modifications are intended to be within the scope of the present invention.

We claim:

1. A method for prophylaxis and/or therapy of dermal damage comprising administering to an individual a composition comprising a toll like receptor (TLR) agonist, wherein the administration is performed prior to, concurrent with or subsequent to the dermal damage.

2. The method of claim 1, wherein the administering is selected from topical, subcutaneous, or systemic administration of the composition comprising the TLR agonist.

3. The method of claim 2, wherein the administering is a topical or a subcutaneous administration.

4. The method of claim 1, wherein the TLR agonist is selected from a TLR2 agonist, a TLR5 agonist, and a combination thereof.

5. The method of claim 4, wherein the TLR5 agonist is CBLB502, and wherein the TLR2 agonist is CBLB612 or CBLB613.

6. The method of claim 5, wherein the dermal damage is a result of radiation, or non-radiation thermal damage, or a cut or puncture.

7. The method of claim 6, wherein the dermal damage comprises non-radiation thermal damage, or a cut or puncture.

8. The method of claim 7, wherein the administration is a topical or subcutaneous administration.

9. The method of claim 6, wherein the prophylaxis and/or therapy results in inhibition of inflammation.

10. The method of claim 9, wherein the TLR5 agonist is CBLB502.

11. A pharmaceutical or cosmetic or nutraceutical formulation suitable for topical or subcutaneous administration, the pharmaceutical or cosmetic formulation comprising a toll like receptor (TLR) agonist.

12. The pharmaceutical or cosmetic or nutraceutical formulation of claim 11, wherein the TLR agonist is selected from a TLR2 agonist, a TLR5 agonist, and a combination thereof

13. The pharmaceutical or cosmetic or nutraceutical formulation of claim 12, wherein the TLR5 agonist is CBLB502, and wherein the TLR2 agonist is CBLB612 or CBLB613.

14. An article of manufacture comprising a dermal damage healing aid, wherein the dermal damage healing aid is coated and/or impregnated with a TLR agonist.

15. The article of manufacture of claim 14, wherein the TLR agonist is selected from a TLR2 agonist, a TLR5 agonist, and a combination thereof, and wherein the dermal damage healing aid is selected from a medical dressing, a suture, a bandage, and a fabric mesh.
